(19) United States
(12) Patent Application Publication
(10) Pub. No.: US 2025/0312101 A1
(43) Pub. Date: Oct. 9, 2025

(54) AUTOMATIC ALIGNMENT OF ANKLE PROTHESES

(71) Applicant: Howmedica Osteonics Corp., Mahwah, NJ (US)

(72) Inventors: Cody Allen Kairis, Eagan, MN (US); Dean Justin Nachtrab, Memphis, TN (US); Chad Michael Kilgore, Eagan, MN (US)

(21) Appl. No.: 19/170,848

(22) Filed: Apr. 4, 2025

Related U.S. Application Data
(60) Provisional application No. 63/631,202, filed on Apr. 8, 2024.

(51) Int. Cl. A61B 34/10; A61F 2/42; G06T 19/20

(52) U.S. Cl. CPC A61B 34/10; A61F 2/4202; G06T 19/20; A61B 2034/105; A61B 2034/108; A61F 2002/4205; A61F 2002/4207; G06T 2210/41; G06T 2219/2004

(57) ABSTRACT

A computer-implemented method comprises, in response to receiving an indication of user input to change a total ankle replacement (TAR) prosthesis system, determining a position and orientation of a tibial prosthesis model relative to a tibial bone model, the tibial prosthesis model being a 3-dimensional virtual model of the tibial prosthesis; determining a position of a talar prosthesis model relative to a talar bone model, the second talar prosthesis model being a 3-dimensional virtual model of the second talar prosthesis; and outputting, for display at a display device, the second tibial prosthesis model at the determined position and orientation of the second tibial prosthesis model relative to the tibial bone model and the second talar prosthesis model at the determined position and orientation of the second talar prosthesis model relative to the talar bone model.

This patent application claims the benefit of U.S. Provisional Patent Application 63/631,202, filed Apr. 8, 2024, the entire content of which is incorporated by reference.

### BACKGROUND

In a total ankle replacement (TAR) surgery, a distal portion of a tibia of a patient's ankle joint and a proximal portion of a talus of the patient's ankle joint are replaced with a tibial prosthesis and a talar prosthesis, respectively. The tibial prosthesis and the talar prosthesis have complementary articulating surfaces that slide relative to one another. In some instances, the tibial prosthesis includes a tibial tray that is attached to the tibia and a polyethylene insert component connected to the tibial tray. In such instances, an articulating surface of the talar prosthesis slides relative to the polyethylene insert component. The articulating surfaces of the tibial prosthesis and the talar prosthesis take the place of damaged or worn natural articulating surfaces of the tibia and talus, which may reduce pain and restore a natural range of motion.

Because different people have different anatomical characteristics, a surgeon may need to choose among different sizes and types of tibial and talar prostheses. The surgeon may also need to choose how to position the tibial and talar prostheses relative to the tibia and the talus.

### SUMMARY

This disclosure describes techniques for computer-assisted preoperative planning of total ankle replacement (TAR) surgeries. As described herein, a surgical planning system may allow a user (e.g., a surgeon) to select among a plurality of different types of TAR prosthesis systems. The types of TAR prosthesis systems may include a TAR prosthesis system with a stemmed tibial prosthesis and a TAR prosthesis system with a stemless tibial prosthesis. If a TAR surgery has been planned using a first TAR prosthesis system and the surgical planning system receives an indication of user input to plan the TAR surgery using a second TAR prosthesis system, the surgical planning system may automatically update at least one of a planned position or orientation of at least one of the tibial or talar prosthesis of the second TAR prosthesis system relative to the tibia and talus. In other words, the user may not need to manually update the position or orientation of the tibial or talar prosthesis when the TAR prostheses changes. Automatically updating the planned position or orientation may increase the efficiency of the surgical planning system in planning the TAR surgery.

In one example, this disclosure describes a computer-implemented method comprising: receiving, by one or more processors implemented in circuitry, an indication of user input to change a total ankle replacement (TAR) prosthesis system from a first TAR prosthesis system to a second TAR prosthesis system, the first TAR prosthesis system including a first tibial prosthesis and a first talar prosthesis and the second TAR prosthesis system including a second tibial prosthesis and a second talar prosthesis; and in response to receiving the indication of user input to change the TAR prosthesis system: determining, by the one or more processors, a position and orientation of a second tibial prosthesis model relative to a tibial bone model, the tibial bone model being a 3-dimensional virtual model of a tibia of an ankle joint of a patient, the second tibial prosthesis model being a 3-dimensional virtual model of the second tibial prosthesis; determining, by the one or more processors, a position of a second talar prosthesis model relative to a talar bone model, the talar bone model being a 3-dimensional virtual model of a talus of the ankle joint of the patient, the second talar prosthesis model being a 3-dimensional virtual model of the second talar prosthesis; and outputting, by the one or more processors, for display at a display device, the second tibial prosthesis model at the determined position and orientation of the second tibial prosthesis model relative to the tibial bone model and the second talar prosthesis model at the determined position of the second talar prosthesis model relative to the talar bone model.

In another example, this disclosure describes a computing system comprising: a memory; and one or more processors implemented in circuitry and communicatively coupled to the memory, the one or more processors configured to receive an indication of user input to change a total ankle replacement (TAR) prosthesis system from a first TAR prosthesis system to a second TAR prosthesis system, the first TAR prosthesis system including a first tibial prosthesis and a first talar prosthesis and the second TAR prosthesis system including a second tibial prosthesis and a second talar prosthesis; and in response to receiving the indication of user input to change the TAR prosthesis system: determine a position and orientation of a second tibial prosthesis model relative to a tibial bone model, the tibial bone model being a 3-dimensional virtual model of a tibia of an ankle joint of a patient, the second tibial prosthesis model being a 3-dimensional virtual model of the second tibial prosthesis; determine a position of a second talar prosthesis model relative to a talar bone model, the talar bone model being a 3-dimensional virtual model of a talus of the ankle joint of the patient, the second talar prosthesis model being a 3-dimensional virtual model of the second talar prosthesis; and output, for display at a display device, the second tibial prosthesis model at the determined position and orientation of the second tibial prosthesis model relative to the tibial bone model and the second talar prosthesis model at the determined position of the second talar prosthesis model relative to the talar bone model.

In another example, this disclosure describes one or more non-transitory computer-readable media having instructions stored thereon that, when executed by one or more processors of a computing system, cause the computing system to receive an indication of user input to change a total ankle replacement (TAR) prosthesis system from a first TAR prosthesis system to a second TAR prosthesis system, the first TAR prosthesis system including a first tibial prosthesis and a first talar prosthesis and the second TAR prosthesis system including a second tibial prosthesis and a second talar prosthesis; and in response to receiving the indication of user input to change the TAR prosthesis system: determine a position and orientation of a second tibial prosthesis model relative to a tibial bone model, the tibial bone model being a 3-dimensional virtual model of a tibia of an ankle joint of a patient, the second tibial prosthesis model being a 3-dimensional virtual model of the second tibial prosthesis; determine a position of a second talar prosthesis model relative to a talar bone model, the talar bone model being a 3-dimensional virtual model of a talus of the ankle joint of the patient, the second talar prosthesis model being a 3-dimensional virtual model of the second talar prosthesis; and output, for display at a display device, the second tibial prosthesis model at the determined position and orientation of the second tibial prosthesis model relative to the tibial bone model and the second talar prosthesis model at the determined position of the second talar prosthesis model relative to the talar bone model.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a conceptual diagram illustrating an example computing system in which one or more techniques of this disclosure may be performed.

FIG. 2 is a conceptual diagram illustrating an example user interface showing differences between alignment to a mechanical axis of a tibia and alignment to an anatomical axis of the tibia, in accordance with one or more techniques of this disclosure.

FIG. 3 is a conceptual diagram illustrating an example user interface showing an example stemless tibial prosthesis model, in accordance with one or more techniques of this disclosure.

FIG. 4A and FIG. 4B are conceptual diagrams illustrating an example talar prosthesis.

FIG. 5 is a flowchart illustrating an example operation of a surgical planning system in accordance with one or more techniques of this disclosure.

### DETAILED DESCRIPTION

During a total ankle replacement (TAR) surgery, a surgeon implants a tibial prosthesis on a distal tibia of a patient's ankle joint and a talar prosthesis on a talus of the patient's ankle joint. When planning the TAR surgery, the surgeon may select among different TAR prosthesis systems. Each TAR prosthesis system may include a range of sizes of tibial protheses and a range of sizes of talar protheses designed to operate with the tibial prostheses of the prosthesis system. Different TAR prosthesis systems may include tibial prostheses having different types of anchorage or other physical characteristics. For example, tibial prostheses in a first TAR prosthesis system may be stemless and tibial prostheses in a second prosthesis system may be stemmed. A stemmed tibial prosthesis includes a stem component that extends into the intermedullary canal of the patient's tibia. In contrast, a stemless tibial prosthesis does not include a stem component extending into the intermedullary canal of the patient's tibia. Stemmed tibial prostheses may be advantageous relative to stemless tibial prostheses when the patient has severe arthritis.

A surgical planning system may help a user, such as a surgeon) plan the TAR surgery. For example, the surgical planning system may display a user interface that presents a tibial bone model and a talar bone model. The tibial bone model may be a 3-dimensional virtual model of the tibia. The talar bone model may be a 3-dimensional virtual model of the talus. The user or the surgical planning system may determine a position and orientation of a first tibial prosthesis model relative to the tibial bone model. The first tibial prosthesis model may be a 3-dimensional virtual model of a first tibial prosthesis. Additionally, the surgeon or the surgical planning system may determine a position of a first talar prosthesis model relative to the talar bone model. The first talar prosthesis model may be a 3-dimensional virtual model of a first talar prosthesis. The first tibial prosthesis and the first talar prosthesis belong to a first TAR prosthesis system, such as a TAR prosthesis system having stemmed tibial prostheses or a TAR prosthesis system having stemless tibial prostheses. Each of the tibial bone model, the talar bone model, and first tibial prosthesis model may be a 3-dimensions mesh. These 3-dimensional meshes may comprise hundreds or thousands of vertices, edges, and faces. Greater numbers of vertices, edges, and faces are associated with greater accuracy and precision.

Different prosthesis systems may have different positioning guidelines. For example, a guideline for positioning a stemless tibial prosthesis may specify that the stemless tibial prosthesis is to be rotationally aligned with a mechanical axis of the patient's tibia and that the center of the stemless tibial prosthesis is to be on the mechanical axis of the patient's tibia. In contrast, a guideline for positioning a stemmed tibial prosthesis may specify that the stemmed tibial prosthesis is to be rotationally aligned with an anatomic axis of the intermedullary canal of the patient's tibia and a center of the stemmed tibial prosthesis is to be on the anatomic axis. In accordance with one or more techniques of this disclosure, the surgical planning system may automatically determine orientations and/or positions of tibial prothesis models and talar prosthesis models when the surgical planning system receives an indication of user input to switch between prosthesis systems. The automatic determination of the orientations and/or positions of tibial prosthesis model and talar prosthesis model may increase accuracy of plans for TAR surgeries and reduce surgical planning time.

Additionally, conventional processes for determining orientations and positions of tibial and talar prosthesis models are associated with significant computational resource requirements. For example, conventional processes for determining orientations and positions of tibial and talar prosthesis models typically require the computing system to perform many rotate, zoom, translate, and model-to-model collision detection operations as the user provides input to the computing system to review, analyze, and adjust the positions of the tibial and talar prosthesis models on the computing system following a change of prosthesis systems. Performing such operations is frequently needed because positioning information associated with the positions of the first tibial and talar prosthesis models is lost when changing between prosthesis systems. Since the meshes representing tibial and talar prosthesis models and bone models may include large numbers of vertices, edges, and faces such operations may require more sophisticated hardware to run smoothly, since such operations may involve multiple memory read requests and significant demands on a graphics processing pipeline. Automating the positioning of the tibial prosthesis model and talar prothesis model as described in this disclosure may avoid or reduce the need for performing such operations. Thus, the techniques of this disclosure may allow a surgical planning system to operate on simpler hardware while still allowing for the use of high precision meshes.

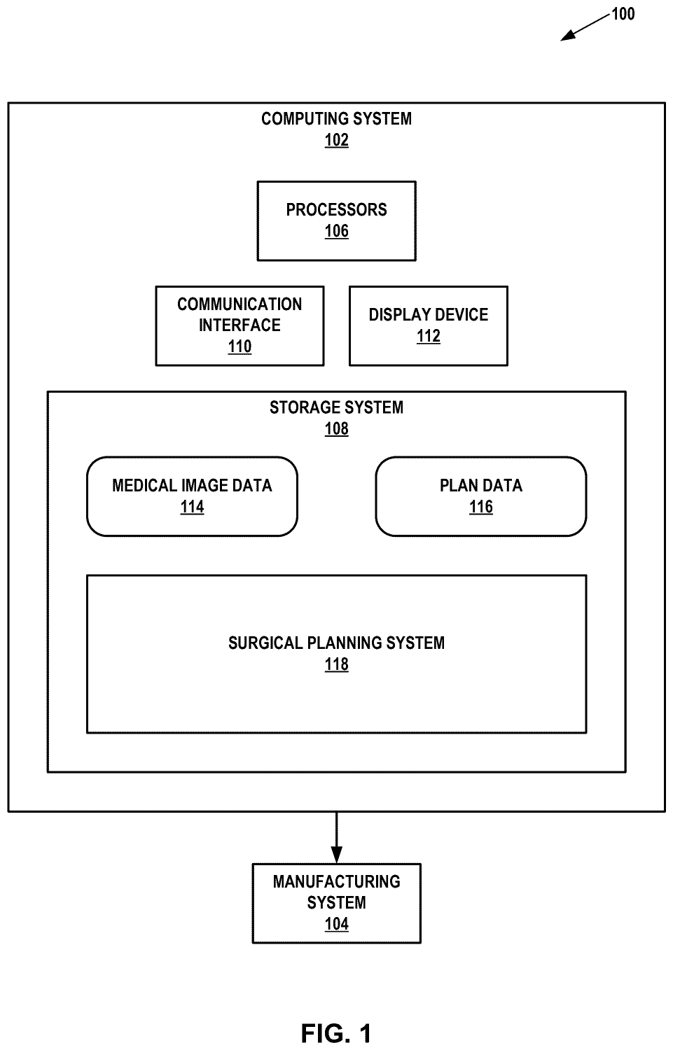

FIG. 1 is a conceptual diagram illustrating an example system 100 in which one or more techniques of this disclosure may be performed. In the example of FIG. 1, system 100 includes a computing system 102 and a manufacturing system 104. Computing system 102 is configured to assist one or more users in generating a surgical plan for an orthopedic surgery, such as a TAR surgery or other type of surgery. Manufacturing system 104 is configured to manufacture patient-specific guides according to surgical plans generated by computing system 102. In some examples, system 100 does not include manufacturing system 104.

Computing system 102 may include one or more computing devices. In one example, computing system 102 includes a personal computer used by a surgeon. In this example, the personal computer may generate a surgical plan without interaction with other computing devices. In other examples, computing system 102 includes a server device and a client device (e.g., a personal computer). In such examples, the server device may generate a surgical plan based on input initially received via the client device. In any case, one or more computing devices of computing system 102 may output user interfaces for display to a user and may receive, directly or indirectly, indications of user input.

Computing system 102 includes one or more processors 106, a storage system 108, a communication interface 110, and a display device 112. In other examples, computing system 102 may include more, fewer, or different components. The components of computing system 102 may be in one or more computing devices. For example, processors 106 may be in a single computing device or distributed among multiple computing devices of computing system 102, storage system 108 may be in a single computing device or distributed among multiple computing devices of computing system 102, and so on. In some examples, computing system 102 is a personal computer, a system of computing devices, one or more server devices, or a system comprising one or more other types of computing devices. Processors 106, storage system 108, communication interface 110, and display device 112 are communicatively coupled.

Processors 106 may be implemented in circuitry and include one or more microprocessors, digital signal processors (DSPs), application specific integrated circuits (ASICs), field programmable gate arrays (FPGAs), hardware, or any combinations thereof. In general, processors 106 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, one or more of the units may be distinct circuit blocks (fixed-function or programmable), and in some examples, the one or more units may be integrated circuits.

Processors 106 may include arithmetic logic units (ALUs), elementary function units (EFUs), digital circuits, analog circuits, and/or programmable cores, formed from programmable circuits. In examples where the operations of processors 106 are performed using software executed by the programmable circuits, storage system 108 may store the object code of the software that processors 106 receives and executes, or another memory within processors 106 (not shown) may store such instructions. Examples of the software include software designed for surgical planning. Processors 106 may perform the actions ascribed in this disclosure to the software.

Processors 106 may output data (e.g., a user interface, models, etc.) for display. Outputting data for display may include one or more of processors 106 generating and sending signals to a display device (e.g., display device 112) that the display device can directly use to display the data. Outputting data for display may include one or more of processors 106 outputting data for transmission to another computing device (e.g., another computing device of computing system 102) that processes the data to generate signals that a display device (e.g., display device 112) may directly use to display the data.

Processors 106 may receive indications of user input from one or more users. Processors 106 may receive an indication of user input directly from a user input device (e.g., keyboard, mouse, touchscreen, etc.). For instance, in an example where computing system 102 is implemented on a single computing device, processors 106 may receive the indications of user input from one or more user input device of the computing device. In other examples, processors 106 may receive the indications of user input by way of one or more computing devices. For instance, in an example where computing system 102 is implemented using a server device and a client device and processors 106 are located in the server device, processors 106 may receive indications of the user input from the client device. For example, processors 106 may receive an indication from the client device that the user has selected a displayed element, typed specific text, and so on.

Storage system 108 may store various types of data used by processors 106. Storage system 108 may include any of a variety of memory devices, such as dynamic random access memory (DRAM), including synchronous DRAM (SDRAM), magnetoresistive RAM (MRAM), resistive RAM (RRAM), or other types of memory devices. Examples of display device 112 include a liquid crystal display (LCD), a plasma display, an organic light emitting diode (OLED) display, or another type of display device.

Communication interface 110 allows computing system 102 to output data and instructions to and receive data and instructions from a medical imaging system, manufacturing system 104, or other devices via one or more communication links or networks. Communication interface 110 may include hardware circuitry that enables computing system 102 to communicate (e.g., wirelessly or using wires) to other computing systems and devices. Example networks may include various types of communication networks including one or more wide-area networks, such as the Internet, local area networks, and so on. In some examples, the network may include wired and/or wireless communication links.

In the example of FIG. 1, storage system 108 stores medical image data 114, plan data 116, and a surgical planning system 118. In other examples, storage system 108 may store more, fewer, or different types of data or units. Moreover, the data and units illustrated in the example of FIG. 1 are provided for purposes of explanation and may not represent how data is actually stored or how software is actually implemented. Surgical planning system 118 may comprise instructions that are executable by processors 106. For ease of explanation, this disclosure may describe surgical planning system 118 as performing various actions when processors 106 execute instructions of surgical planning system 118.

Surgical planning system 118 is a system that may help a surgeon plan an orthopedic surgery as part of a pre-operative planning process. Surgical planning system 118 may be an instance of a computer-assisted orthopedic surgery (CAOS) system or a computer-assisted surgical system (CASS). In some examples, surgical planning system 118 may also assist users during a surgery. For instance, surgical planning system 118 may output navigation information to one or more devices (e.g., monitors, head-mounted displays, etc.) to a user during a surgery to help the user execute a surgical plan.

During the pre-operative planning process, surgical planning system 118 may output a user interface for display to a user. The user of surgical planning system 118 may be a surgeon, technician, or other type of user. The user interface may present a tibial bone model and a talar bone model. The tibial bone model may be a 3-dimensional virtual model of the tibia. The talar bone model may be a 3-dimensional virtual model of the talus. The surgeon or the surgical planning system may determine a position and orientation of a tibial prosthesis model relative to the tibial bone model. The surgeon or the surgical planning system may determine a position and orientation of a talar prosthesis model relative to the talar bone model. The tibial prosthesis model may be a 3-dimensional virtual model of a tibial prosthesis. The talar prosthesis model may be a 3-dimensional virtual model of a talar prosthesis. Models, such as the tibial bone model, talar bone model, talar prosthesis model, and tibial prosthesis model, may comprise meshes. Each of the meshes may include a set of vertices, edges, and faces. Each of the meshes may include thousands of such vertices, edges, and faces.

In some examples, a tibial bone model and talar bone model may be generated (e.g., by surgical planning system 118 or another system) based on medical image data 114. Medical image data 114 may include a computed tomography (CT) scan and/or other type of medical imaging of the patient's ankle. For example, surgical planning system 118 may receive CT slices of the patient's ankle, convert the CT slices into one or more 3D images, and perform a segmentation process on the one or more 3D images to determine shapes of the distal tibial and the talus. In some examples, surgical planning system 118 may also receive x-ray or CT images of the patient's entire tibia, potentially including the patient's distal femur. Images of the patient's entire tibia may allow surgical planning system 118 to determine a mechanical axis of the patient's tibia. In some examples, the user interface of surgical planning system 118 may display 2-dimensional (2D) models of the patient's distal tibia, talus, and/or other bones.

Additionally, the user interface of surgical planning system 118 may include features (e.g., buttons, drop-down boxes, radio buttons, menus, etc.) that enable the user to select a TAR prosthesis system from among a plurality of TAR prosthesis systems for use in TAR surgeries. For example, the user interface of surgical planning system 118 may include features that enable the user to select a TAR prosthesis system from among a TAR prosthesis system that includes stemmed tibial prostheses and a TAR prosthesis system that includes stemless tibial protheses. Surgical planning system 118 may reposition and reorient the tibial prosthesis model and talar prosthesis model in response to indications of user input from the user. In this way, surgical planning system 118 may enable the user to select appropriate positions and orientations of the tibial prosthesis and the talar prosthesis.

At some point during planning of the TAR surgery, the user may decide to switch from one TAR prosthesis system to another TAR prosthesis system. For example, the user may decide to switch from a TAR prosthesis system having stemless tibial prostheses to a TAR prosthesis system having stemmed tibial prostheses. For example, the user may determine, after positioning the stemless tibial prosthesis model relative to the tibial bone model, that there would not be enough cortical bone in the tibia to adequately support a stemless tibial prosthesis, that the distance between the stemless tibial prosthesis model and an outer edge of a medial or lateral malleolus of the tibia is below a threshold. In this case, the user may decide to switch to a TAR prosthesis having the stemmed tibial prostheses. There may be other reasons for switching as well.

Conversely, the user may decide to switch from a TAR prosthesis system having stemmed tibial prostheses to a prosthesis system having stemless tibial prostheses. For example, the user, after positioning the stemmed tibial prosthesis model, may decide that a stemless tibial prosthesis would be sufficient and a stemmed tibial prosthesis is not needed. In general, implantation of a stemless tibial prosthesis is less invasive than implantation of a stemmed tibial prosthesis.

Accordingly, the user may use the features provided by the user interface of surgical planning system 118 to switch from one TAR prosthesis system to another TAR prosthesis system. In other words, surgical planning system 118 may receive an indication of user input to switch from one TAR prosthesis system to another TAR prosthesis system.

In response to receiving an indication of user input to switch from a first TAR prosthesis system to a second TAR prosthesis system, surgical planning system 118 may automatically determine a position and orientation of a second tibial prosthesis model relative to the tibial bone model. Surgical planning system 118 may automatically determine the position and orientation of a prosthesis model in the sense that the user does not need to manually determine the position and orientation of the prosthesis model. The second tibial prosthesis model may be a 3-dimensional virtual model of the second tibial prosthesis of the second TAR prosthesis system. Additionally, surgical planning system 118 may automatically determine a position of a second talar prosthesis model relative to the talar bone model. The second talar prosthesis model may be a 3-dimensional virtual model of the second talar prosthesis of the second TAR prosthesis system. Surgical planning system 118 may output, for display at display device 112, the second tibial prosthesis model at the determined position and orientation of the second tibial prosthesis model relative to the tibial bone model and the second talar prosthesis model at the determined position of the second talar prosthesis model relative to the talar bone model.

As part of determining the position and orientation of a tibial prosthesis model, surgical planning system 118 may determine a tibial landmark position. The tibial landmark position may correspond to a center of a distal surface of the tibia. In some examples, surgical planning system 118 receives an indication of user input to specify the tibial landmark position. In some examples, surgical planning system 118 may automatically determine the tibial landmark position, e.g., as described in PCT publication WO 2023/239513, filed May 9, 2023, the entire content of which is incorporated by reference. Surgical planning system 118 may determine the position of the tibial prosthesis such that a center of a distal surface of the tibial prosthesis model coincides (i.e., is collocated) with the tibial landmark position. In this way, tibial landmark position may serve to establish a proximal-distal position of the tibial prosthesis model. Furthermore, as part of determining the position and orientation of the second tibial prosthesis model after switching to the second TAR prosthesis system, surgical planning system 118 may determine the position of the second tibial prosthesis such that a center of a distal surface of the second tibial prosthesis model coincides with the tibial landmark position. In this way, despite the tibial prostheses of different TAR prosthesis systems potentially having different proximal-distal heights, the distal surfaces of the tibial prostheses may still be the same. In other words, the joint line of the ankle may be maintained when the TAR prosthesis system changes.

In some examples, surgical planning system 118 may automatically determine a size of a talar prosthesis model. For instance, surgical planning system 118 may automatically determine the size of the talar prosthesis model in response to receiving an indication of user input to change the TAR prosthesis system. In some examples, to automatically determine the size of the talar prosthesis, surgical planning system 118 may determine a talar landmark position corresponding to a center of a talar dome of the talus. Surgical planning system 118 may determine a proximal-distal position of the talar prosthesis model such that a center of a talar dome of the talar prosthesis model coincides with the talar landmark position. Surgical planning system 118 may determine a plane through the talar bone model corresponding to a distal surface of the talar prosthesis model when the talar prosthesis is at the determined proximal-distal position. An anterior-posterior length of the plane is a distance between an anterior intersection point of the plane and an anterior edge of the talar bone model and a posterior intersection point of the plane and a posterior edge of the talar bone model. Surgical planning system 118 may determine an anterior-posterior size of the talar prosthesis model based on the anterior-posterior length of the plane. For instance, surgical planning system 118 may determine the anterior-posterior size of the talar prosthesis model as the size closest to the anterior-posterior length of the plane.

In some examples, as part of automatically determining the position of a tibial bone model, surgical planning system 118 may determine an axis of the tibia. Surgical planning system 118 may determine an anterior-posterior position of the tibial prosthesis model to center the tibial prosthesis on the axis. For instance, if the tibial prosthesis is a stemless tibial prosthesis, the axis may be a mechanical axis of the tibia. Surgical planning system 118 may automatically determine the mechanical axis of the tibia. For instance, to automatically determine the mechanical axis of the tibia, surgical planning system 118 may identify a landmark on a proximal surface of the tibia and a landmark on a distal surface of the tibia. In some examples, surgical planning system 118 may automatically determine the landmarks on the proximal and distal surfaces of the tibia, e.g., as described in PCT publication WO 2023/239513. Surgical planning system 118 may determine the mechanical axis as a line between the landmark on the proximal surface of the tibia and the landmark on the distal surface of the tibia.

In some examples where the tibial prosthesis is a stemless tibial prosthesis, surgical planning system 118 may automatically determine the orientation of the tibial prosthesis model. As part of automatically determining the orientation of the tibial prosthesis model, surgical planning system 118 may determine, based at least in part on the tibial bone model, a mechanical axis of the tibia and determine the orientation of the tibial prosthesis model based on the mechanical axis of the tibia. As part of determining the orientation of the tibial prosthesis model based on the mechanical axis of the tibia, surgical planning system 118 may determine a coronal rotation of the tibial prosthesis model such that a line orthogonal to a medial-lateral axis of the tibial prosthesis model is aligned with the mechanical axis. Additionally, surgical planning system 118 may determine a sagittal rotation of the tibial prosthesis such that a line orthogonal to an anterior-posterior axis of the tibial prosthesis model is aligned with the mechanical axis. Surgical planning system 118 may rotate the tibial prosthesis model around a centroid of a bounding box surrounding the tibial prosthesis model. Rotation of the tibial prosthesis model around this centroid may minimize anterior-posterior and medial-lateral shift of the tibial prosthesis model when rotating the tibial prosthesis model. Rotation of a model, such as the tibial prosthesis model, may involve updating the coordinates of each vertex of a mesh representing the model.

If the tibial prosthesis is a stemmed tibial prosthesis, surgical planning system 118 may determine the anterior-posterior position of the tibial prostheses model to coincide with a center of the tibial prosthesis on an anatomical axis of the tibia. The anatomic axis of the tibia is an imaginary line passing through the intermedullary canal of the tibia. Surgical planning system 118 may automatically determine the anatomical axis of the tibia. For example, surgical planning system 118 may determine a landmark on a distal surface of the tibia, e.g., as described above. Additionally, surgical planning system 118 may determine a landmark located within the intermedullary canal of the tibia at a specific distance (e.g., 80 mm, 90 mm, etc.) from the landmark at the distal end of the tibia. Surgical planning system 118 may determine the anatomical axis of the tibia as an imaginary line connecting the landmark on the distal surface of the tibia and the landmark located within the intermedullary canal of the tibia. Thus, surgical planning system 118 may determine the orientation of the tibial prosthesis model based on the anatomic axis of the tibia.

In some examples where the tibial prosthesis is a stemmed tibial prosthesis, surgical planning system 118 may automatically determine the orientation of the stemmed tibial prosthesis model. As part of automatically determining the orientation of the stemmed tibial prosthesis model, surgical planning system 118 may determine, based at least in part on the tibial bone model, the anatomical axis of the tibia and may determine the orientation of the stemmed tibial prosthesis model based on the anatomical axis of the tibia. As part of determining the orientation of the stemmed tibial prosthesis model based on the anatomical axis of the tibia, surgical planning system 118 may determine a coronal rotation of the stemmed tibial prosthesis model such that a line orthogonal to a medial-lateral axis of the tibial prosthesis model is aligned with the anatomical axis. Additionally, surgical planning system 118 may determine a sagittal rotation of the stemmed tibial prosthesis such that a line orthogonal to an anterior-posterior axis of the stemmed tibial prosthesis model is aligned with the anatomical axis. Surgical planning system 118 may rotate the stemmed tibial prosthesis model around a centroid of a bounding box surrounding the stemmed tibial prosthesis model. Rotation of the stemmed tibial prosthesis model around this centroid may minimize anterior-posterior and medial/lateral shift of the stemmed tibial prosthesis model when rotating the stemmed tibial prosthesis model.

In some examples where the tibial prosthesis is a stemless tibial prosthesis, surgical planning system 118 may automatically determine the position of the tibial prosthesis model. As part of automatically determining the position of the tibial prosthesis model, surgical planning system 118 may determine a tibial landmark position corresponding to a center of a distal surface of the tibia. Surgical planning system 118 may determine a proximal-distal position of the tibial prosthesis model based on the tibial landmark position. For instance, surgical planning system 118 may set the proximal-distal position of the tibial prosthesis model such that a point on a distal surface of the tibial prosthesis model coincides with the tibial landmark position. Additionally, surgical planning system 118 may determine a plane through the tibial bone model corresponding to a proximal surface of the talar prosthesis model when the tibial prosthesis model is at the determined proximal-distal position. An anterior-posterior length of the plane is a distance between an anterior intersection point of the plane and an anterior edge of the tibial bone model and a posterior intersection point of the plane and a posterior edge of the tibial bone model. Surgical planning system 118 may determine an anterior-posterior position of the tibial prosthesis model to minimize a distance between an anterior edge of the second tibial prosthesis model and the anterior intersection point of the plane.

Surgical planning system 118 may generate plan data 116 that describes a plan for a TAR surgery for a specific patient. Plan data 116 may include data specifying the types and sizes of tibial and talar prostheses, their positions, and their orientation.

As mentioned above, manufacturing system 104 is configured to manufacture patient-specific guides according to surgical plans generated by computing system 102. The patient-specific guide is a physical object that helps surgeons cut bones (e.g., the tibia and talus) during the TAR surgery. For example, patient-specific guide may assist the surgeon in cutting the tibia at a plane aligned with the proximal, lateral, and medial edges of the tibial prosthesis model. Thus, after the position and orientation of the tibial prosthesis model has been determined, computing system 102 may output data that specify to manufacturing system 104 the positions of the proximal, lateral, and medial edges of the tibial prosthesis model, and manufacturing system 104 may generate the patient-specific guide accordingly. Manufacturing the patient-specific guide may involve an additive manufacturing process, such as a 3D printing process. A patient-specific surface of the patient-specific guide may need to match a surface of the patient's bone with high precision. For example, the patient-specific surface of the patient-specific guide may need high precision in order to accommodate osteophytes, lesions, or other irregularities in the patient's bone. In order to provide for such high precision, the meshes representing the bones and prosthesis models may also need to have high precision. As discussed above, performing operations on high precision meshes may impose significant computational burdens on computing systems. Thus, precision of the meshes may be lowered to accommodate computing requirements. This may lead to lower-accuracy patient-specific guides. The techniques of this disclosure may reduce computational burdens associated with the operations and therefore allow for higher precision meshes (e.g., even for hardware with limited computational capability). Hence, the techniques of this disclosure may allow for higher precision patient-specific guides. Higher precision patient-specific guides may seat better on the patient's anatomy, potentially enabling a surgeon to conduct a surgery with greater precision.

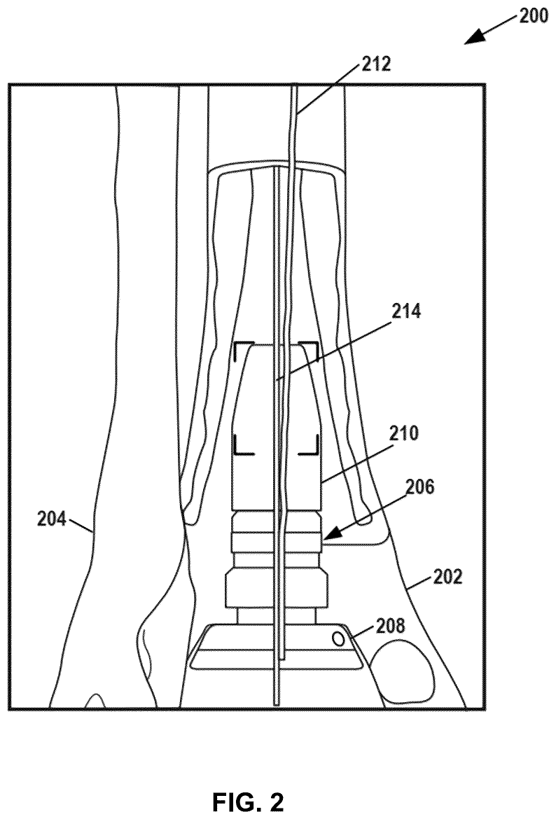

FIG. 2 is a conceptual diagram illustrating an example user interface 200 showing differences between alignment to a mechanical axis of a tibia and alignment to an anatomical axis of the tibia, in accordance with one or more techniques of this disclosure. In the example of FIG. 2, user interface 200 shows a tibial bone model 202, a fibular bone model 204, and a stemmed tibial prosthesis model 206. Stemmed tibial prosthesis model 206 that includes a base component model 208 and a stem component model 210. Surgical planning system 118 may update a position and/or orientation of stemmed tibial prosthesis model 206 relative to tibial bone model 202 in response to indications of user input.

User interface 200 also shows a mechanical axis 212 of the tibia and an anatomical axis 214 of the tibia. Surgical planning system 118 may receive an indication of user input to switch a TAR prosthesis system from a first TAR prosthesis system to a second TAR prosthesis system. In response, surgical planning system 118 may determine an orientation of a tibial prosthesis model of a tibial prosthesis of the second TAR prosthesis system. In the example of FIG. 2, the tibial prosthesis of the second TAR prosthesis system is a stemmed tibial prosthesis. Accordingly, surgical planning system 118 may determine an orientation of stemmed tibial prosthesis model 206 such that stemmed tibial prosthesis model 206 is aligned with anatomical axis 214. Surgical planning system 118 also shows mechanical axis 212 to show to the user the difference in orientation that resulted from switching from the first TAR prosthesis system to the second TAR prosthesis system.

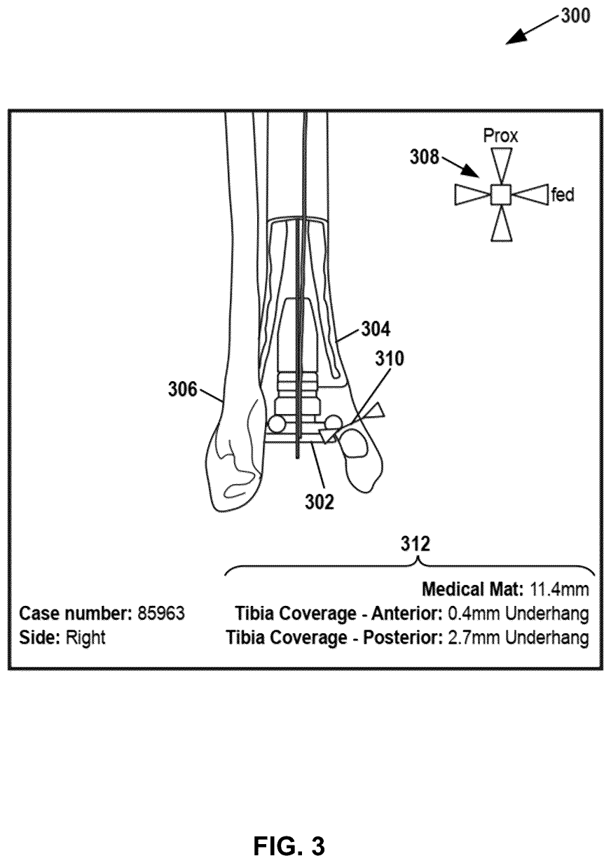

FIG. 3 is a conceptual diagram illustrating an example user interface 300 showing an example stemless tibial prosthesis model 302, in accordance with one or more techniques of this disclosure. In the example of FIG. 3, user interface 300 shows a tibial bone model 304, a fibular bone model 306, and stemless tibial prosthesis model 302. User interface 300 also includes position controls 308. Surgical planning system 118 may update the position of stemless tibial prosthesis model 302 in response to indications of user input to position controls 308. In the example of FIG. 3, a polyethylene insert component (not shown) may be subsequently connected to stemless tibial prosthesis model 302 to form an articulation surface for stemless tibial prosthesis model 302.

User interface 300 also includes other information. For example, user interface 300 includes an element 310 indicating a distance between a medial edge of stemless tibial prosthesis model 302 and a medial edge of the medial malleolus of the tibial bone model 304. Additionally, user interface 300 includes elements 312 that provide information about the position of stemless tibial prosthesis model 302 relative to tibial bone model 304. Specifically, elements 312 provide information about an anterior coverage of stemless tibial prosthesis model 302 and a posterior coverage of stemless tibial prosthesis model 302. The anterior coverage of stemless tibial prosthesis model 302 indicates a length by which an anterior edge of stemless tibial prosthesis model 302 extends beyond (overhangs) an anterior edge of tibial bone model 304 or a length by which the anterior edge of tibial bone model 304 extends beyond (underhangs) the anterior edge of stemless tibial prosthesis model 302. The posterior coverage of stemless tibial prosthesis model 302 indicates a length by which a posterior edge of stemless tibial prosthesis model 302 extends beyond (overhangs) a posterior edge of tibial bone model 304 or a length by which the posterior edge of tibial bone model 304 extends beyond (underhangs) the posterior edge of stemless tibial prosthesis model 302. User interface 200 may have information similar to elements 312.

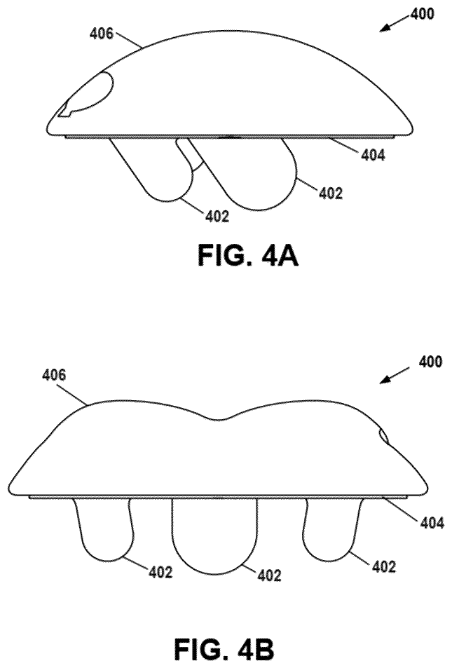

FIG. 4A and FIG. 4B are conceptual diagrams illustrating an example talar prosthesis 400. FIG. 4A shows talar prosthesis 400 from an anterior perspective. FIG. 4B shows talar prosthesis 400 from a lateral perspective. Talar prosthesis 400 has a set of pegs 402 extending distally from a distal surface 404 of talar prosthesis. A proximal surface 406 of talar prosthesis 400 may articulate relative to a tibial prosthesis (or an articulating insert component thereof).

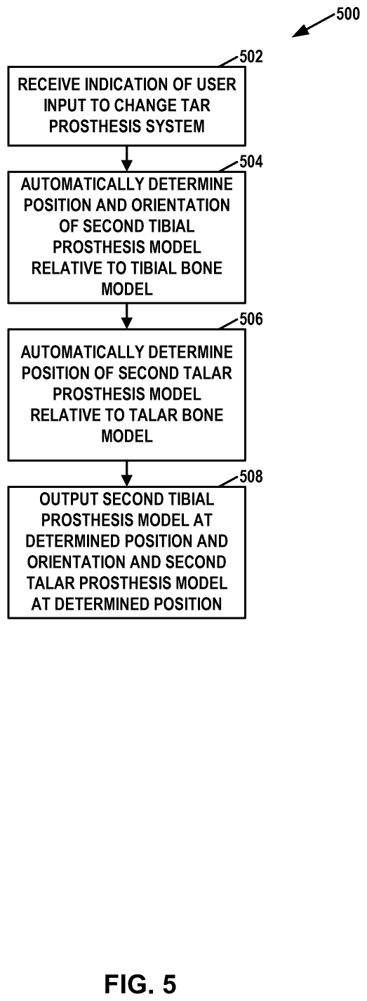

FIG. 5 is a flowchart illustrating an example operation 500 of surgical planning system 118 in accordance with one or more techniques of this disclosure. In the example of FIG. 5, surgical planning system 118 may receive an indication of user input to change a TAR prosthesis system from a first TAR prosthesis system to a second TAR prosthesis system (502). The first TAR prosthesis system includes a first tibial prosthesis and a first talar prosthesis. The second TAR prosthesis system includes a second tibial prosthesis and a second talar prosthesis. Surgical planning system 118 may receive the indication of user input to change the TAR prosthesis system via a user interface.

In response to receiving the indication of user input to change the TAR prosthesis system, surgical planning system 118 may automatically determine a position and orientation of the second tibial prosthesis model relative to a tibial bone model (504). The tibial bone model is a 3-dimensional virtual model of a tibia of an ankle joint of a patient. The second tibial prosthesis model is a 3-dimensional virtual model of the second tibial prosthesis. As part of determining the position of the second tibial prosthesis model, surgical planning system 118 may determine a proximal-distal position of the second tibial prosthesis model. Surgical planning system 118 may determine the proximal-distal position of the second tibial prosthesis model such that a point on an articulating surface of the second tibial prosthesis model coincides with a distal tibia landmark. The distal tibia landmark may correspond to a center of a distal surface of the tibia. The same distal tibia landmark may also coincide with a point on the articulating surface of the first tibial prosthesis model.

As part of determining the position of the second tibial prosthesis model, surgical planning system 118 may determine an anterior-posterior position of the second tibial prosthesis model. In examples where the second tibial prosthesis is a stemless tibial prosthesis, surgical planning system 118 may determine a plane through the tibial bone model corresponding to a proximal surface of the second talar prosthesis model when the second tibial prosthesis model is at the determined proximal-distal position. Surgical planning system 118 may determine an anterior-posterior position of the second tibial prosthesis model to minimize a distance between an anterior edge of the second tibial prosthesis model and the anterior intersection point of the plane.

As part of determining the orientation of the second tibial prosthesis model, surgical planning system 118 may determine an axis of the tibia (e.g., a mechanical axis or an anatomical axis of the tibia) and determine the orientation of the second tibial prosthesis model based on the axis.

Furthermore, surgical planning system 118 may automatically determine a position of a second talar prosthesis model relative to a talar bone model (506). The talar bone model is a 3-dimensional virtual model of a talus of an ankle joint of a patient. The second talar prosthesis model is a 3-dimensional virtual model of the second talar prosthesis. For example, to determine a proximal-distal position of the second talar prosthesis model such that a point on a proximal surface of the second talar prosthesis model coincides with a point on a proximal surface of the talus. In some examples, surgical planning system 118 may determine the point on the proximal surface of the talus. Surgical planning system 118 may determine a proximal-distal position of a first talar prosthesis model such that a point on a proximal surface of the first talar prosthesis model coincides with the point on the proximal surface of the talus, the first talar prosthesis model being a 3-dimensional representation of the first talar prosthesis. Thus, the joint line of the ankle joint may be maintained when the TAR prosthesis system is switched but cut lines through the talus corresponding to distal surfaces of the first and second talar prostheses may change because the first and second talar prostheses may have different proximal-distal heights.

Surgical planning system 118 may output, for display at display device 112, the second tibial prosthesis model at the determined position and orientation of the second tibial prosthesis model relative to the tibial bone model and the second talar prosthesis model at the determined position of the second talar prosthesis model relative to the talar bone model (508). For example, surgical planning system 118 may output a user interface, such as user interface 200 or user interface 300, that shows the second tibial prosthesis model, the second talar prosthesis model, the tibial bone model, and the talar bone model. The user interface may also show other bone models, such as a fibular bone model and/or bone models corresponding to a calcaneus or other bones. To output the second tibial prosthesis model at the determined position and orientation relative to the tibial bone model and the second talar prosthesis model at the determined position of the second talar prosthesis model relative to the talar bone model may comprise performing a rendering process that generates a 2-dimensional image based on meshes of the second tibial prosthesis model, the tibial bone model, and the second talar prosthesis model. The rendering process may involve the use of a shader program to compute pixel or vertex colors, a rasterization process to render the 3D mesh onto a 2-dimensional plane, post-processing, and so on. In contrast to previous techniques where a computing system would need to perform rendering process multiple times as the user makes adjustments to the positioning and orientation of the prosthesis models and adjusts the rotation and zoom levels to do so, surgical planning system 118 may, in accordance with the techniques of this disclosure, only need to perform this rendering process once. Thus, computational requirements may be reduced, potentially while maintaining high precision meshes.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuitry,” as used herein may refer to any of the foregoing structures or any other structure suitable for implementation of the techniques described herein.

What is claimed is:

1. A computer-implemented method comprising:
receiving, by one or more processors implemented in circuitry, an indication of user input to change a total ankle replacement (TAR) prosthesis system from a first TAR prosthesis system to a second TAR prosthesis system, the first TAR prosthesis system including a first tibial prosthesis and a first talar prosthesis and the second TAR prosthesis system including a second tibial prosthesis and a second talar prosthesis; and
in response to receiving the indication of user input to change the TAR prosthesis system:
determining, by the one or more processors, a position and orientation of a second tibial prosthesis model relative to a tibial bone model, the tibial bone model being a 3-dimensional virtual model of a tibia of an ankle joint of a patient, the second tibial prosthesis model being a 3-dimensional virtual model of the second tibial prosthesis;
determining, by the one or more processors, a position of a second talar prosthesis model relative to a talar bone model, the talar bone model being a 3-dimensional virtual model of a talus of the ankle joint of the patient, the second talar prosthesis model being a 3-dimensional virtual model of the second talar prosthesis; and
outputting, by the one or more processors, for display at a display device, the second tibial prosthesis model at the determined position and orientation of the second tibial prosthesis model relative to the tibial bone model and the second talar prosthesis model at the determined position of the second talar prosthesis model relative to the talar bone model.

2. The method of claim 1, wherein:
the determining the position and orientation of the second tibial prosthesis model comprises:
determining, by the one or more processors, a tibial landmark position corresponding to a center of a distal surface of the tibia; and
determining, by the one or more processors, the position of the second tibial prosthesis model such that a center of a distal surface of the second tibial prosthesis model coincides with the tibial landmark position.

3. The method of claim 1, wherein the determining the position of the second tibial prosthesis model comprises:
determining, by the one or more processors, an axis of the tibia; and
determining, by the one or more processors, an anterior-posterior position of the second tibial prosthesis model to center the second tibial prosthesis on the axis.

4. The method of claim 3, wherein the second tibial prosthesis is a stemless tibial prosthesis and the axis is a mechanical axis of the tibia.

5. The method of claim 3, wherein the second tibial prosthesis is a stemmed tibial prosthesis and the axis is an anatomical axis of the tibia.

6. The method of claim 1, wherein:
the second tibial prosthesis is a stemless tibial prosthesis, and
the determining the position of the second tibial prosthesis model comprises:
determining, by the one or more processors, a tibial landmark position corresponding to a center of a distal surface of the tibia;
determining, by the one or more processors, a proximal-distal position of the second tibial prosthesis model based on the tibial landmark position;
determining, by the one or more processors, a plane through the tibial bone model corresponding to a proximal surface of the second tibial prosthesis model when the second tibial prosthesis model is at the determined proximal-distal position; and
determining, by the one or more processors, an anterior-posterior position of the second tibial prosthesis model to minimize a distance between an anterior edge of the second tibial prosthesis model and an anterior intersection point, the anterior intersection point being a point of intersection of the plane and an anterior edge of the tibial bone model.

7. The method of claim 1, wherein:
the first tibial prosthesis is a stemmed tibial prosthesis and the second tibial prosthesis is a stemless tibial prosthesis, and
the determining the orientation of the second tibial prosthesis model comprises:
determining, by the one or more processors, based at least in part on the tibial bone model, a mechanical axis of the tibia; and
determining, by the one or more processors, the orientation of the second tibial prosthesis model based on the mechanical axis of the tibia.

8. The method of claim 7, wherein the determining the orientation of the second tibial prosthesis model comprises:
determining, by the one or more processors, a coronal rotation of the second tibial prosthesis model such that a line orthogonal to a medial-lateral axis of the second tibial prosthesis model is aligned with the mechanical axis; and
determining, by the one or more processors, a sagittal rotation of the second tibial prosthesis such that a line orthogonal to an anterior-posterior axis of the second tibial prosthesis model is aligned with the mechanical axis.

9. The method of claim 1, wherein:
the first tibial prosthesis is a stemless tibial prosthesis and the second tibial prosthesis is a stemmed tibial prosthesis, and
determining the orientation of the second tibial prosthesis model comprises:
determining, by the one or more processors, based at least in part on the tibial bone model, an anatomic axis of the tibia; and
determining, by the one or more processors, the orientation of the second tibial prosthesis model based on the anatomic axis of the tibia.

10. The method of claim 1, wherein:
the first talar prosthesis and the second talar prosthesis have different proximal-distal heights, and
determining the position of the second talar prosthesis model relative to the talar bone model comprises determining, by the one or more processors, a proximal-distal position of the second talar prosthesis model such that a point on a proximal surface of the second talar prosthesis model coincides with a point on a proximal surface of the talus.

11. The method of claim 10, wherein a first talar prosthesis model representing the first talar prosthesis is positioned such that a point on a proximal surface of the first talar prosthesis model coincides with the point on the proximal surface of the talus.

12. The method of claim 1, further comprising, prior to receiving the indication of user input to change the TAR prosthesis system:
determining, by the one or more processors, a position and orientation of a first tibial prosthesis model relative to the tibial bone model, the first tibial prosthesis model being a 3-dimensional virtual model of the first tibial prosthesis; and
determining, by the one or more processors, a position of a first talar prosthesis model relative to the talar bone model, the first talar prosthesis model being a 3-dimensional virtual model of the first talar prosthesis.

13. A computing system comprising:
a memory; and
one or more processors implemented in circuitry and communicatively coupled to the memory, the one or more processors configured to:
receive an indication of user input to change a total ankle replacement (TAR) prosthesis system from a first TAR prosthesis system to a second TAR prosthesis system, the first TAR prosthesis system including a first tibial prosthesis and a first talar prosthesis and the second TAR prosthesis system including a second tibial prosthesis and a second talar prosthesis; and
in response to receiving the indication of user input to change the TAR prosthesis system:
determine a position and orientation of a second tibial prosthesis model relative to a tibial bone model, the tibial bone model being a 3-dimensional virtual model of a tibia of an ankle joint of a patient, the second tibial prosthesis model being a 3-dimensional virtual model of the second tibial prosthesis;
determine a position of a second talar prosthesis model relative to a talar bone model, the talar bone model being a 3-dimensional virtual model of a talus of the ankle joint of the patient, the second talar prosthesis model being a 3-dimensional virtual model of the second talar prosthesis; and
output, for display at a display device, the second tibial prosthesis model at the determined position and orientation of the second tibial prosthesis model relative to the tibial bone model and the second talar prosthesis model at the determined position of the second talar prosthesis model relative to the talar bone model.

14. The computing system of claim 13, wherein the one or more processors are configured to, as at least part of the determining the position and orientation of the second tibial prosthesis model:
determine a tibial landmark position corresponding to a center of a distal surface of the tibia; and
determine the position of the second tibial prosthesis model such that a center of a distal surface of the second tibial prosthesis model coincides with the tibial landmark position.

15. The computing system of claim 13, wherein the one or more processors are configured to, as at least part of the determining the position of the second tibial prosthesis model:
determine an axis of the tibia; and
determine an anterior-posterior position of the second tibial prosthesis model to center the second tibial prosthesis on the axis.

16. The computing system of claim 13, wherein:
the second tibial prosthesis is a stemless tibial prosthesis, and
the one or more processors are configured to, as at least part of the determining the position of the second tibial prosthesis model:
determine a tibial landmark position corresponding to a center of a distal surface of the tibia;
determine a proximal-distal position of the second tibial prosthesis model based on the tibial landmark position;
determine a plane through the tibial bone model corresponding to a proximal surface of the second tibial prosthesis model when the second tibial prosthesis model is at the determined proximal-distal position; and
determine an anterior-posterior position of the second tibial prosthesis model to minimize a distance between an anterior edge of the second tibial prosthesis model and an anterior intersection point, the anterior intersection point being a point of intersection of the plane and an anterior edge of the tibial bone model.

17. The computing system of claim 13, wherein:
the first tibial prosthesis is a stemmed tibial prosthesis and the second tibial prosthesis is a stemless tibial prosthesis, and
the one or more processors are configured to, as at least part of the determining the orientation of the second tibial prosthesis model:
determine, based at least in part on the tibial bone model, a mechanical axis of the tibia; and
determine the orientation of the second tibial prosthesis model based on the mechanical axis of the tibia.

18. The computing system of claim 13, wherein:
the first tibial prosthesis is a stemless tibial prosthesis and the second tibial prosthesis is a stemmed tibial prosthesis, and
the one or more processors are configured to, as at least part of determining the orientation of the second tibial prosthesis model:
determine, based at least in part on the tibial bone model, an anatomic axis of the tibia; and
determine the orientation of the second tibial prosthesis model based on the anatomic axis of the tibia.

19. The computing system of claim 13, wherein:
the first talar prosthesis and the second talar prosthesis have different proximal-distal heights, and
the one or more processors are configured to, as at least part of the determining the position of the second talar prosthesis model relative to the talar bone model, determine a proximal-distal position of the second talar prosthesis model such that a point on a proximal surface of the second talar prosthesis model coincides with a point on a proximal surface of the talus.

20. One or more non-transitory computer-readable media having instructions stored thereon that, when executed by one or more processors of a computing system, cause the computing system to:
receive an indication of user input to change a total ankle replacement (TAR) prosthesis system from a first TAR prosthesis system to a second TAR prosthesis system, the first TAR prosthesis system including a first tibial prosthesis and a first talar prosthesis and the second TAR prosthesis system including a second tibial prosthesis and a second talar prosthesis; and
in response to receiving the indication of user input to change the TAR prosthesis system:
determine a position and orientation of a second tibial prosthesis model relative to a tibial bone model, the tibial bone model being a 3-dimensional virtual model of a tibia of an ankle joint of a patient, the second tibial prosthesis model being a 3-dimensional virtual model of the second tibial prosthesis;
determine a position of a second talar prosthesis model relative to a talar bone model, the talar bone model being a 3-dimensional virtual model of a talus of the ankle joint of the patient, the second talar prosthesis model being a 3-dimensional virtual model of the second talar prosthesis; and
output, for display at a display device, the second tibial prosthesis model at the determined position and orientation of the second tibial prosthesis model relative to the tibial bone model and the second talar prosthesis model at the determined position of the second talar prosthesis model relative to the talar bone model.
